SEQUENCE LISTING (5 sequences)

SEQ ID NO: 1 - DNA, 1538 bp, Bacillus licheniformis
agagtttgatcctggctcaggacgaacgctggcggcgtgcctaatacatgcaagtcgagcggacagatgggagcttgctccctgatgtyagcggcggacgggtgagtaacacgtgggtaacctgcctgtaagactgggataactccgggaaaccggggctaataccggatgcttgattgaaccgcatggttcaattataaaaggtggcttttagctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaagccgcgaggctaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttggagccagccgccgaaggtgggacagatgattggggtgaagtcgtaacaaggtagccgtatcggaaggtgcggctggatcacct

SEQ ID NO: 2 - DNA, 1197 bp, Bacillus licheniformis
gtgaagaataaatggctttcttttttttcaggaaagatccagcttgagataacgggaaaggggatcgagcgattattaaatgaatgcaccaggcgcagcatcccgatgtttaatgttaagaaaaagaaagacgctgtctttctttatattccgctttctgatgtacatgccttccggagggtcataagggattttgactgcaagtgccgcttcgtcagacgaaaaggtttccctttccttgtgcagaagtctaaacggaatagcggcttcactattggaattgctgctttttttatcatcatgtttttattgtccaacatgctctggaaaattgacataacaggagccaatccggagacagaacatcaaatcagacagcacttgaatcaaatcggcgtcaaaaaagggcgccttcagtttacgatgctgaccccggaaaaaattcagcaggcgcttacaaagcgcgtcgaaaacatcacctgggtgggcattgaattaaacggcacggcccttcacatgaaagtggttgaaaaaaatgaacctgacaaagaaaaatatattggtccgaggcacatcgtcgccaaaaaaggggcgaccatctcgaaaatgttcgtggaaaaaggcgagccgctcgtcaccgtgaatcagcacgttgaaaaagggcaaatgcttgtttccggactgatcgggagcgaagaggaaaagcaaaaagtcggagccaaagggaaaatctacggtgaaacctggtacaagtcaaaggtaacggttcctcttgagacatcatttgacgtttttacgggtaaagtaaggacaagtcacaagctatccttcggttctttttctgtgcctgtctggggcttttcttttaaaaaagaagagttctcgcgtccgaagacagaaaccgaaaaacactcgctgcattttatgaattttacgcttcctgtcgcgtatgaaaaggaacatacgagagagagcgaacaaatcaaaagggtgtactcgaaaaaagaagcggtccttgaaggggtcgaaatgggaaaaagagacatcaggaaaaaaatcgggagcgacgggaacattattagtgaaaaagttttgcacgaaacgagtgagaatggtaaagttaaattgatcatcctttaccaggttattgaagacattgttcaaacaacaccaattgttcaggagactaaagaatga

SEQ ID NO: 3 - DNA, 1920 bp, Bacillus licheniformis
gtggcaatggaacagcaaaataattacgatgaaaatcagatacaggttctagagggattggaagccgtgcggaaaagacccggaatgtacatcgggtcgacaagcggcaaaggtctgcaccatttagtatgggaaattgtcgataacagcatcgatgaagcattggccggttactgcactgaaatcaatgtcgaaattgaaaaagacaacagcatcactgtaaaagacaacggacgggggattccggtcggtattcatgagaagatgggccgtcccgctgtagaagtcatcatgacagtcctgcacgccggaggaaagtttgacggaagcggatataaagtttcaggcggtttgcacggcgtcggtgcatctgttgttaacgccctttcaaccgagctcgatgtaacggtttacagagatggaaaagtccattaccaggaatttgaacggggcgttccgaaagctgatttgaaagtcatcggagatacggaagtgacgggaacgaccactcacttcaagcctgatccggaaatattcacggaaacgacggaatacgactatgatacgcttgccactcgtgtccgggagctcgctttcttgacaaaaggcgtcaaaatcacgattgaagacaagcgagaaggaaaagaacgcaagaatgattactgctatgaaggcggtattaaaagctatgttgaacacttgaaccgttcacgggaagtggttcatgaagagccagtctatattgaaggatccaaagacggcattacggtcgaggtggctcttcaatacaacgacagttataccagcaacatttattcgtttgccaataacattcatacgtatgaaggcggaacgcatgaagccggctttaagaccggtttgacgagagtcatcaatgattacgcgagaaggaacggtgtcttcaaagaaagcgatccgaacttaagcggggaagacgtccgtgaaggtttgacagcgatcatttcaatcaagcatccggatcctcaatttgaagggcagacgaaaacaaagcttggcaactcagaagcgcggacgataacagatgcgctattttcagaagcgctcgaaaagtttctgcttgaaaacccggattcggcgaaaaaaatcgttgaaaaaggggttatggccgccagagcacgaatggctgcaaagaaagcacgcgaactgacgcgcagaaaaagcgcccttgaagtgtcgaatctgccggggaaactggctgactgttcttctaaagacccgacgatttccgaactttacatcgttgagggtgactctgcgggcggatcggcaaaacagggccgcgatcgtcatttccaagccattttgcctttgagagggaaaatcttgaacgtcgaaaaagcacgcctggacaaaattttgtccaacaatgaggttcgttctatgatcaccgcgcttggcaccgggatcggggaagatttcaatcttgaaaaagcccgctaccacaaagtcgtgattatgacggatgccgacgtcgacggtgcccacatccgaacgttgctgctgacctttttctaccgctacatgagacagatcatcgaacaaggatatgtctacatcgctcagccgccgctttacaaaatacagcaggggaaacgcgttgagtatgcgtacaatgaccagcagctcgaagaaattctaaaagagcttccgcaaaatccgaagccaggcctgcagcgctataaaggtttgggcgagatgaacgcgacacagctttgggaaacgacgatggaccctgaaacgcggacattgcttcaagtaacgctcaaagatgcgatcgatgcggatgaaacctttgaaatgctgatgggcgacaaggttgagcctcgccgcaactttatcgaagagaatgcccgatatgtaaagaatttggatatttaa

SEQ ID NO: 4 - DNA, 3582 bp, Bacillus licheniformis
ttgacaggtcaactagttcagtatggacgacaccgccagcgcagaagctatgcacgcattagcgaagtgttagaattaccaaatctcattgaaattcaaacctcttcttatcagtggtttcttgatgagggtcttagagagatgtttcaagacatatcgccaattgaggatttcactggtaacctctctctggaatttatcgactacagcttgggcgagcctaagtatccggtagaagaatcaaaagagcgcgacgtgacctactcagctccgctgcgtgttaaagtccgcttaatcaacaaagagaccggtgaagtaaaggatcaggatgtcttcatgggtgatttccctattatgacagacactggaacctttattatcaacggtgcggaacgggtcattgtatctcagctcgttcgttctccaagtgtatattttagtggtaaagtagacaaaaacggtaagaaaggttttaccgcgactgtcattccaaaccgtggcgcatggttagaatacgagactgatgcgaaagatgttgtttatgtacgcatcgatcgcacacgtaagttgccggttacggttcttttgcgtgctcttggcttcggatctgaccaagagatcattgatctcatcggtgaaaacgagtatttgcgcaatacgcttgataaagataatacggaaaacaccgataaagcgcttcttgaaatctacgagcgtcttcgtccaggagagccgcctacagtagaaaacgcaaaaagcctgcttgattcaaggttctttgatccgaaaagatatgaccttgcaagtgtaggacgttataaaattaataaaaagcttcacatcaaaaacagactgtttaatcagcggcttgctgaaacgctgatcgatcctgaaacaggcgaaattcttgccgaaaaaggcgcgattttagacagaagaacgcttgacaaagtcctgccttaccttgaaaacggaatcggttttaaaaagctgtatccgaacggcggagttgttgaagatgaagtgacgcttcagtctattaaaatctatgccccgacagatcaagaaggggagcagacaatcaatgtgattggaaatgcttatattgaagaaggcgttaaaaatattacaccttctgatattatctcttccatcagctatttctttaacctgcttcacggagtgggcgataccgacgatatcgaccatttaggaaaccgccgtctccgttcagtgggagagcttctgcaaaaccaattccgtatcggtttaagcagaatggagcgtgttgttcgtgaaagaatgtctattcaagatacaaacacgatcacgccacagcagctgatcaatattcgccctgtcatcgcatcaatcaaagagtttttcggaagctcgcagctttctcagtttatggatcagacgaatccgcttgctgagctgacgcataagcgccgtctgtcagcgctcggaccgggcggtttgacccgtgagcgcgccggaatggaagtccgtgacgttcactattcccactacggccggatgtgtccgattgaaactcctgagggtccaaacatcggcttgatcaactcgctttcttcattcgcgaaagtgaaccgtttcggcttcatcgaaacgccgtatcgccgcgttgaccctgagactggaaaagtaacgccgagaatcgattacttgacagctgatgaagaagacaactatgtcgttgcccaggctaacgcacgtctgaatgatgacggatcttttgtggatgacagcatcgtcgcccgtttcagaggggagaacaccgttgttccgaaagaccgcgtcgactatatggacgtttcgcctaaacaggttgtctctgccgcgactgcatgtattcctttcttggaaaacgacgactcaaaccgcgccctaatgggagcgaacatgcaacggcaggctgtacctcttatgcagcctgaatctccgatcgtcggaaccggaatggagtatgtatctgcaaaagactccggtgccgctgttatttgccgccatcctggaatcgttgaacgggtggaagcgaaaaacatctgggtgcgccgctatgaagaagtcgacggccagaaagtcaaaggaaaccttgataaatacagcctgctgaagtttgttcgttccaaccagggaacttgctacaaccagcgtccgatcgtaagcgtcggtgatgaggttgtaaaaggtgaaattttagctgacggtccgtctatggaaaaaggtgagcttgcccttggacgcaacgtcatggtcggctttatgacatgggatggctacaactacgaggatgccatcatcatgagcgaacgccttgtaaaagacgacgtatacacgtctattcatattgaggaatacgaatcagaggcccgggatacaaaactcggacctgaagaaattacacgcgatattccaaacgtcggtgaagacgctcttcgcaatctcgatgaacgcggaattatccgtgtcggtgctgaagtaaaagacggagatcttcttgttggtaaagtaacgcctaaaggtgttacagagcttactgcagaagagcgcctgcttcacgccatcttcggcgaaaaagcgcgtgaagtccgcgatacgtcgctgcgtgtgccccacggaggcggcggtatcatccttgatgtaaaagtgttcaaccgcgaagacggagacgaactgcctccgggcgttaaccagctcgtccgcgtttacatcgttcagaaacgtaaaatttctgaaggggacaaaatggccggacgccacgggaacaaaggtgttatttcgaaaatccttccggaggaagatatgccgtatctgcctgacggaacaccgattgacatcatgttaaacccgctgggcgtaccatcgcgtatgaacatcgggcaggtgttggagctgcaccttggtatggctgcacgccgtctcggtctgcatgtcgcgtcgccggtatttgacggtgcccgcgaagaagatgtgtgggaaacgcttgaagaagccggcatgtcaagggacgcaaagacagtcctttacgacggccgaactggagagccgtttgacaaccgggtgtctgtcggcatcatgtacatgatcaaattggctcacatggttgacgacaaattgcacgcgcgttctacaggtccttactcactcgttacccagcagcctcttggaggtaaagcgcagttcggtggacagcgttttggagagatggaagtttgggcgcttgaagcttacggcgcagcatatacacttcaagaaatcctgactgttaaatcggatgatgtcgtaggccgtgtgaaaacatacgaagccatcgtaaaaggcgacaatgttccagaacctggtgttccggaatcgttcaaagtattgatcaaagagcttcaaagcttaggtatggacgtcaaaatcctatcaagcgacgaagaagaaatcgaaatgagagacttggaagacgacgaagacgcgaaacaaaacgaagggctttctctgccgaatgatgaagagtccgaagaattcgtttctgctgacgcagagcgcgatgtcgtcacaaaagaataa

SEQ ID NO: 5 - DNA, 1635 bp, Bacillus licheniformis
atggcaaaagatattaagtttagcgaagaagcccgccgttcaatgctgcgcggtgtagatgcattggcggatgctgtaaaggtaactttgggacctaaaggacgcaacgtcgttcttgagaaaaaattcggttctccattaatcacaaacgacggtgttaccatcgcgaaagaaatcgaacttgaagacgcgttcgaaaacatgggcgcaaagcttgttgctgaagttgcgagcaaaacaaacgatgttgccggtgacggtacaacaacagcgacagttctagctcaagcgatgattcgcgaaggtcttaaaaacgtaactgccggcgctaaccctgtaggcgtgcgcaaaggtatcgagcaggctgtggctgtagcggttgaaagcctgaaagaaatctccaaaccaatcgaaggcaaagaatcaatcgcacaagttgcttcaatctccgctgctgacgaagaagtcggaagcctgatcgctgaagcgatggagcgcgtcggcaacgacggtgtcatcacgatcgaagaatcaaaaggattcacaacagagcttgaagtggttgaaggtatgcagttcgaccgcggatatgcgtctccttacatggtgacagattccgataagatggaagcggttcttgagaacccgtacatcttagtaacagacaaaaaaatcacaaacattcaagaaatcctgccggtgcttgagcaagttgttcagcaaggcaaaccgttgcttctgattgctgaagacgttgaaggtgaggctcttgcaacattggttgtcaacaagcttcgcggaacattcaacgcagtggctgttaaagcgcctggattcggagaccgccgcaaagcgatgctcgaagacatctcgatcctgacaggtgccgaagtgatcacagaagatttaggtcttgaccttaaatcaactcaaatcaaccaattgggacgcgcttctaaagttgttgtgacaaaagaaaacacaacaatcgttgaaggtgcgggagatacagagcaaatcgcggcacgcgtcaaccaaatccgcgctcaagttgaagaaacaacttctgagttcgataaagaaaaattacaagagcgcctagctaagcttgcaggcggcgtagctgtcatcaaagtcggcgctgcgactgaaacagaattgaaagaacgcaagcttcgcatcgaagacgccctgaactctactcgcgctgcggtagaagaaggaatcgtatcaggcggtggtacagctcttgtcaatgtttacaataaagtagctgcccttgaagctgaaggcgacgaactgactggtatcaacatcgttcttcgcgctcttgaagaaccaatccgtcaaatcgcgcacaatgcgggtcttgaaggatctgtcatcgttgagcgcctgaaaggcgaagctattggtgtaggctacaacgctgcaacaggcgaatgggtgaacatgatcgacaaaggtatcgttgaccctacaaaagtaacacgctcagctctgcaaaacgcagcatctgtagcggctatgttcctgacaactgaagcagttgttgccgacaagcctgaagaaaacaaaggcggcgcaggaatgcctgacatgggcggcatgggtggtatgggcggcatgatgtaa

(19) United States
(12) Patent Application Publication
(10) Pub. No.: US 2021/0228653 A1
(43) Pub. Date: Jul. 29, 2021

(54) BACILLUS LICHENIFORMIS STRAIN WITH PROBIOTIC ACTIVITY

(71) Applicant: EVONIK DEGUSSA GMBH, Essen (DE)

(72) Inventors: Daniel Petri, Wien (AT); Stefan PELZER, Gütersloh (DE); Jessica KLEINBÖLTING, Bielefeld (DE); Stella MOLCK, Bielefeld (DE); Maike KIPKER, Köln (DE); Claudia BORGMEIER, Bensheim (DE); Sandra HERBOLD, Mannheim (DE); Guido MEURER, Seeheim-Jugenheim (DE); Rose WHELAN, Offenbach (DE); Kiran DORANALLI, Frankfurt (DE)

(73) Assignee: EVONIK DEGUSSA GMBH, Essen (DE)

(21) Appl. No.: 16/305,811

(22) PCT Filed: May 24, 2017
(86) PCT No.: PCT/EP2017/062495

§ 371 Date: Nov. 29, 2018

(30) Foreign Application Priority Data
May 31, 2016 (EP) 16172151.9
Jun. 3, 2016 (CN) 201610564646.1
Oct. 27, 2016 (EP) 16196015.8

(51) Int. Cl. A61K 35/742; C12N 1/24; A01N 63/22; C12R 1/10

(52) U.S. Cl. CPC A61K 35/742; C12R 1/10; A01N 63/22; C12N 1/24

(57) ABSTRACT

The current invention concerns a new B. licheniformis strain with strong inhibition of C. perfringens and its use as probiotic.

The current invention concerns a new B. licheniformis strain with strong inhibition of C. perfringens and its use as probiotic.

The use of B. licheniformis strains as probiotic ingredient in the feed industry is well known in the state of the art. The function of probiotics (also called “direct-fed microbials” or “DFM”) is to influence the gut microflora in a positive way by supporting the growth of beneficial bacteria and/or the suppression of the growth of pathogenic bacteria. Ideally, by using probiotics the use of antibiotic growth promotors (APGs) becomes redundant. But besides that, it is desirable that the probiotic fulfills further functions like helping in the digestion of specific feed ingredients.

Thus in view of the state of the art, there is a need for probiotics which influence the gut microflora in a positive way and beyond that desirably fulfill at least one further function.

Surprisingly it was found that the bacteria according to the current invention exhibit many advantageous features. Besides their ability to inhibit growth of C. perfringens, the main commercially relevant pathogen of poultry, they in particular show a very high proliferation rate in presence of bile and help to digest cellulose in a very effective way.

Bacillus licheniformis DSM 32314 has been identified by screening of naturally occurring isolates. It has been deposited with the DSMZ on May 12, 2016 under the provisions of the Budapest Treaty on the International Recognition of the Deposit of Microorganisms for the Purpose of Patent Procedure under the Accession Number as mentioned before in the name of Evonik Degussa GmbH. Based upon its phylogenetic and phenotypic characteristics, the B. licheniformis strain DSM 32314 might also be classified as B. paralicheniformis (s. Dunlap et al. (2015), Int J Syst Evol Microbiol 65, 3487-3492).

Thus a first subject of the current invention is a Bacillus licheniformis strain and/or a preparation of said Bacillus licheniformis strain selected from the following group:

a) The Bacillus licheniformis strain as deposited under DSM 32314 at the DSMZ;

b) a mutant of the Bacillus licheniformis strain as deposited under DSM 32314 having all identifying characteristics of the strain DSM 32314, wherein said mutant preferably has a DNA sequence identity to the strain DSM 32314 of at least 95%, preferably at least 96, 97 or 98%, more preferably at least 99 or 99.5%;

c) a preparation of (a) or (b);

d) a preparation containing an effective mixture of metabolites as contained in (a), (b) or (c).

The Bacillus licheniformis strain as deposited under DSM 32314 at the DSMZ exhibits the following characterizing sequences:

a) a 16S rDNA sequence with a sequence identity of at least 99.5%, above all 100%, to the polynucleotide sequence according to SEQ ID NO: 1;

b) a yqfD sequence with a sequence identity of at least 99.5%, above all 100%, to the polynucleotide sequence according to SEQ ID NO: 2;

c) a gyrB sequence with a sequence identity of at least 99.5%, above all 100%, to the polynucleotide sequence according to SEQ ID NO: 3;

d) an rpoB sequence with a sequence identity of at least 99.5%, above all 100%, to the polynucleotide sequence according to SEQ ID NO: 4;

e) a groEL sequence with a sequence identity of at least 99.5%, above all 100%, to the polynucleotide sequence according to SEQ ID NO: 5.

Thus, a further subject of the current invention is a Bacillus licheniformis strain or a preparation thereof, in particular a B. licheniformis strain with the characteristics as mentioned before, exhibiting at least one, preferably all, of the following characteristics:

a) a 16S rDNA sequence with a sequence identity of at least 99%, preferably at least 99.5%, more preferably at least 99.8 or 99.9%, above all 100%, to the polynucleotide sequence according to SEQ ID NO: 1;

b) a yqfD sequence with a sequence identity of at least 99%, preferably at least 99.5%, more preferably at least 99.8 or 99.9%, above all 100%, to the polynucleotide sequence according to SEQ ID NO: 2;

c) a gyrB sequence with a sequence identity of at least 99%, preferably at least 99.5%, more preferably at least 99.8 or 99.9%, above all 100%, to the polynucleotide sequence according to SEQ ID NO: 3.

Preferably, this B. licheniformis strain exhibits at least one, more preferably all, of the following further characteristics:

d) an rpoB sequence with a sequence identity of at least 99%, preferably at least 99.5%, more preferably at least 99.8 or 99.9%, above all 100%, to the polynucleotide sequence according to SEQ ID NO: 4;

e) a groEL sequence with a sequence identity of at least 99%, preferably at least 99.5%, more preferably at least 99.8 or 99.9%, above all 100%, to the polynucleotide sequence according to SEQ ID NO: 5.

Thus, a particular subject of the current invention is also a Bacillus licheniformis strain, exhibiting the following characteristics:

a) a 16S rDNA sequence according to SEQ ID NO: 1;

b) a yqfD sequence according to SEQ ID NO: 2;

c) a gyrB sequence according to SEQ ID NO: 3.

Preferably, this B. licheniformis strain exhibits the following further characteristics:

d) an rpoB sequence according to SEQ ID NO: 4;

e) a groEL sequence according to SEQ ID NO: 5.

The strains of the current invention are preferably characterized by at least one, more preferably by all, of the following further features:

They are preferably able to grow under anaerobic conditions. Further, they are preferably able to degrade water-insoluble cellulose under such anaerobic conditions.

They preferably inhibit infectious bacteria, in particular C. perfringens, very effectively. In particular they are preferably characterized by a pathogen clearance of at least 10 mm, more preferably at least 13 mm, in a well diffusion antagonism assay on LBKelly agar plates with respect to C. perfringens type strain ATCC 13124.

The spores are preferably viable at low pHs and preferably survive exposure to pHs as low as 4.0, in particular as low as 3.0, preferably as low as 2.0, for at least one hour.

The strains according to the invention are preferably further characterized by being able to grow in the presence of 0.05 wt.-% acetic acid, 0.05 wt-% propionic acid and/or 0.2 wt.-% lactic acid.

They are preferably further characterized by a cellulase activity of at least 200 mU/mL, more preferably at least 230 mU/mL, above all about 250 mU/mL, a xylanase activity of at least 10 mU/mL, more preferably at least 15 mU/mL, above all about 20 mU/mL, and/or a protease activity of at least 6 mU/mL, more preferably at least 8 mU/mL, above all about 10 mU/mL.

The B. licheniformis strains according to the invention are preferably further characterized by being able to grow in presence of 2 mM bile, more preferably in presence of 4 mM bile. In particular they are preferably characterized by an AUCS performance value of at least 0.3, preferably at least 0.4, above all at least 0.5, in particular about 0.54, and an AUC10 performance value of at least 1.5, preferably at least 1.75, above all at least 2.0, in particular about 2.1, in presence of 2 mM bile, respectively.

In addition, the strains are preferably able to grow under high salt conditions, in particular in presence of 10 wt.-% of NaCl, for at least one day.

Further the strains of the current invention preferably survive the high temperatures necessary for pelleting animal feed, in particular they preferably survive a temperature of 80° C. for at least 20 minutes.

As the strain according to the current invention may also be classified as B. paralicheniformis, another subject of the current invention are also B. paralicheniformis strains and preparations thereof with the characteristics as mentioned before.

Without wishing to be bound by any theory, it is thought that the Bacillus licheniformis strains according to the current invention enhance animal health by a multifaceted mode of action, including the production of metabolites with selective efficacy and the competition with pathogenic bacteria by better consuming the available nutrients, thereby suppressing effective establishment of pathogenic bacteria in the gut.

It is a big advantage of probiotics in comparison to antibiotics, that they do not destroy bacteria indiscriminately nor do they lead to antibiotic resistant strains of pathogenic bacteria. Normally they are able to selectively compete with pathogenic bacteria by production of antimicrobial substances with specific efficacy, and are ideally able to simultaneously enhance the growth and viability of beneficial gut microflora. Further, they are preferably able to stimulate a systemic immune response in the treated animals.

The mutant strains of DSM 32314 of the current invention are preferably spontaneous mutants. The term “spontaneous mutant” refers to mutants that arise from DSM 32314 without the intentional use of mutagens. Such spontaneous mutants may be obtained by classical methods, such as growing the Bacillus licheniformis strain in the presence of UV light or in the presence of a certain antibiotic to which the parent is susceptible and testing any resistant mutants for improved biological activity or improved ability to enhance one or more of the indicia of animal health. Other methods for identifying spontaneous mutants are known to those of ordinary skill in the art. But besides these preferred spontaneous mutants all other kinds of mutants of DSM 32314, like mutants obtained by genetic engineering, are also comprised by the current invention.

One particular embodiment of the current invention are naturally non-occurring mutants of the strain DSM 32314 characterized by the features as mentioned before.

In a preferred embodiment of the current invention, the strains and preparations of the present invention are preferably administered orally to animals or human beings.

Thus, a further subject of the current invention are compositions, such as feedstuffs, foodstuffs, drinking and rearing water as well as therapeutic compositions, containing a B. licheniformis strain and/or a preparation of the current invention.

A further subject of the current invention is also the use of a B. licheniformis strain and/or a preparation of the current invention as a probiotic ingredient (DFM) in feed or food products.

A further subject of the current invention is also the use of a B. paralicheniformis strain and/or a preparation thereof as a probiotic ingredient (DFM) in feed or food products.

Preferred foodstuffs according to the invention are dairy products, in particular yoghurt, cheese, milk, butter and quark.

The cells of the strains of the current invention may be present, in particular in the compositions of the current invention, as spores (which are dormant), as vegetative cells (which are growing), as transition state cells (which are transitioning from growth phase to sporulation phase) or as a combination of at least two, in particular all of these types of cells. In a preferred embodiment, the composition of the current invention comprises mainly or only spores.

The Bacillus licheniformis strains of the current invention and compositions containing them, when administered to animals, preferably enhance the health of such animals and/or improve the general physical condition of such animals and/or improve the feed conversion rate of such animals and/or decrease the mortality rate of such animals and/or increase the survival rates of such animals and/or improve the weight gain of such animals and/or increase the productivity of such animals and/or increase the disease resistance of such animals and/or increase the immune response of such animals and/or establish or maintain a healthy gut microflora in such animals and/or reduce the pathogen shedding through the feces of such animals. In particular the strains and compositions of the current invention might be used to assist in re-establishing a healthy balance of the gut microflora after administration of antibiotics for therapeutic purposes.

A further subject of the current invention is therefore a method of enhancing the health of animals and/or of improving the general physical condition of animals and/or of improving the feed conversion rate of animals and/or of decreasing the mortality rate of animals and/or of increasing the survival rates of animals and/or of improving the weight gain of animals and/or of increasing the productivity of animals and/or of increasing the disease resistance of animals and/or of increasing the immune response of animals and/or of establishing or maintaining a healthy gut microflora in animals and/or of reducing the pathogen shedding through the feces of animals, wherein the strains and/or preparations of the current invention or the compositions of the current invention, which comprise such strain(s), are administered to animals.

A further subject of the current invention is therefore also the use of strains and/or preparations and/or compositions of the current invention for enhancing the health of animals and/or for improving the general physical condition of animals and/or for improving the feed conversion rate of animals and/or for decreasing the mortality rate of animals and/or for increasing the survival rates of animals and/or for improving the weight gain of animals and/or for increasing the productivity of animals and/or for increasing the disease resistance of animals and/or for increasing the immune response of animals and/or for establishing or maintaining a healthy gut microflora in animals and/or for reducing the pathogen shedding through the feces of animals, wherein the strains and/or preparations of the current invention or the compositions of the current invention, which comprise such strain(s), are administered to animals.

A further subject of the current invention are therefore also the strains and preparations of the current invention as mentioned before and the compositions of the current invention, containing those strains, for enhancing the health of animals and/or for improving the general physical condition of animals and/or for improving the feed conversion rate of animals and/or for decreasing the mortality rate of animals and/or for increasing the survival rate of animals and/or for improving the weight gain of animals and/or for increasing the productivity of animals and/or for increasing the disease resistance of animals and/or for increasing the immune response of animals and/or for establishing or maintaining a healthy gut microflora in animals and/or for reducing the pathogen shedding through the feces of animals.

“Increasing the productivity of animals” refers in particular to any of the following: production of more or higher quality eggs, milk or meat or increased production of weaned offspring.

The methods and uses of the strains, preparations and compositions of the current invention can be therapeutic or non-therapeutic. In a particularly preferred embodiment of the current invention, the methods and uses are non-therapeutic, in particular feeding applications.

As the untreated manure of animals due to pathogenic bacteria and other ingredients may have a detrimental environmental effect, in particular with respect to the animals themselves and/or with respect to human beings getting in contact with the manure, which can be avoided by either feeding the animals or directly treating the manure or the bedding of the animals with the strains, compositions or preparations of the current invention, therefore a further subject of the current invention is a method of controlling and/or avoiding detrimental environmental effects of manure or contaminated liquids, the method comprising the step of applying to manure, contaminated liquids, litter, a pit, or a manure pond at least one strain, one preparation and/or one composition according to the current invention. Preferably the composition is applied in liquid form, for example by spraying, or as a powder, for example by strewing.

As detrimental bacteria may have a negative influence on the consistency of litter and in particular may effect a rather fluid or highly fluid litter, which might lead to foot pad lesions of poultry and which can be avoided by feeding the animals with the strains, compositions or preparations of the current invention, therefore a further subject of the current invention is a method of controlling and/or improving the consistency of litter, in particular a method of ensuring a solid consistency of litter and/or a method of avoiding foot pad lesions, the method comprising the step of feeding animals, in particular poultry, with at least one strain, one preparation and/or one composition according to the current invention.

The strains and preparations according to the invention can also be used for improving the quality of water. A further subject of the current invention is therefore also a method of controlling and/or improving the quality of water or aqueous solutions, in particular of drinking water and/or rearing water, comprising the step of applying to water or an aqueous solution at least one strain and/or at least one preparation and/or at least one composition according to the invention.

Further, the strains and preparations according to the invention can also be used for treating microbial diseases of plants. A further subject of the current invention is therefore also a method of treating and/or preventing microbial diseases of plants, in particular of cultivated plants, comprising the step of applying to the plants at least one strain and/or at least one preparation and/or at least one composition according to the invention. The application may be carried out in liquid form, such as by spraying, or in solid form, in particular as a powder.

By using the strains, preparations and compositions of the current invention preferably an improvement of at least one of the features as mentioned before is realized, wherein realization of the feature preferably means an improvement of at least 1%, more preferably of at least 3 or at least 5%, in comparison to an adequate negative control. As negative control averages known in the animal husbandry field may be used, but preferably as negative control animals which are subjected to the same treatment like the animals tested are used, but without administration of the strains and/or preparations of the current invention.

In particular, the strains, preparations and compositions of the current invention may be administered or fed to an animal in an amount effective to inhibit and/or decrease the growth of pathogenic bacteria in the animal gut. Such pathogenic bacteria include Clostridia, Listeria, Salmonella, Enterococci, Staphylococci, Aeromonas, Streptococci, Campylobacter, Escherichia coli, and Vibrio. Relatedly, the methods of the present invention may be used to decrease the amount of pathogenic bacteria shed in animal feces. The methods of the present invention may also be used to maintain or increase the growth of beneficial bacteria, such as lactic acid bacteria, in the animal gut. By decreasing pathogenic bacteria and/or increasing or maintaining beneficial bacteria, the compositions of the present invention are able to maintain an overall healthy gut microflora.

Thus, a further subject of the current invention is a method of inhibiting and/or decreasing the growth of harmful or pathogenic bacteria and/or maintaining and/or increasing the growth of beneficial bacteria in an animal gut, wherein strains and/or preparations and/or compositions of the current invention are administered to animals and wherein the pathogenic bacteria are preferably selected from Clostridia, in particular C. perfringens and C. difficile, Listeria, in particular L. monocytogenes, L. seeligeri and L. welshimeri, Salmonella, in particular S. enterica, S. gallinarum, S. pullorum, S. arizonae, S. typhimurium, S. enteritidis, and S. bongori, Enterococci, in particular E. faecalis, E. faecium and E. cecorum, Staphylococcus, in particular S. aureus, Aeromonas, Streptococci, in particular S. suis and S. gallinaceus, Campylobacter, in particular C. jejuni and C. coli, Escherichia coli, and Vibrio, in particular V. parahemolyticus and V. harveyi, and the beneficial bacteria are preferably selected from lactic acid bacteria, in particular from Lactobacilli, and Bifidobacteria.

In a preferred embodiment of the invention the amount of at least one pathogenic bacterium, in particular the amount of C. perfringens, is reduced by at least 0.5 log, more preferably by at least 1 log, 2 log, or 3 log.

Thus, a further subject of the current invention are also the strains, preparations and compositions of the current invention for inhibiting and/or decreasing the growth of pathogenic bacteria and/or for maintaining and/or increasing the growth of beneficial bacteria in an animal gut, wherein the pathogenic bacteria are preferably selected from Clostridia, in particular C. perfringens and C. difficile, Listeria, in particular L. monocytogenes, L. seeligeri and L. welshimeri, Salmonella, in particular S. enterica, S. gallinarum, S. pullorum, S. arizonae, S. typhimurium, S. enteritidis, and S. bongori, Enterococci, in particular E. faecalis, E. faecium and E. cecorum, Staphylococcus, in particular S. aureus, Aeromonas, Streptococci, in particular S. suis and S. gallinaceus, Campylobacter, in particular C. jejuni and C. coli, Escherichia coli, and Vibrio, in particular V. parahemolyticus and V. harveyi, and the beneficial bacteria are preferably selected from lactic acid bacteria, in particular from Lactobacilli, and Bifidobacteria.

The occurrence and/or increased growth of the pathogenic bacteria does or can lead to the outbreak of certain diseases. For example, the occurrence and/or increased growth of Clostridium perfringens can lead to the outbreak of gut diseases, in particular to the outbreak of necrotic enteritis in poultry. The occurrence and/or increased growth of Clostridium perfringens can also lead to the outbreak of further diseases like bacterial enteritis, gangrenous dermatitis and colangiohepatitis. Even the mildest form of infection by C. perfringens can already be accompanied by diarrhea, which results in wet litter and by that may lead to secondary diseases like foot pad dermatitis.

A further subject of the current invention is therefore also a therapeutic composition comprising the strains and/or compositions of the current invention as mentioned before.

A preferred subject in this context is therefore a therapeutic composition for treatment and/or prevention of necrotic enteritis, in particular sub-clinical necrotic enteritis, in animals, preferably poultry, comprising the strains and/or compositions of the current invention as mentioned before. Another preferred subject in this context is therefore a therapeutic composition for treatment and/or prevention of bacterial enteritis, gangrenous dermatitis, colangiohepatitis, clostridiosis, diarrhea and/or foot pad dermatitis, in animals, preferably poultry, comprising the strains and/or compositions of the current invention as mentioned before.

A further subject of the current invention is therefore also the treatment and/or prevention of a disease, in particular of a gut disease, preferably of necrotic enteritis, in particular of sub-clinical necrotic enteritis, in poultry, wherein a strain and/or composition and/or preparation of the current invention is administered to an animal in need thereof.

A further subject of the current invention is therefore also the treatment and/or prevention of a disease, preferably a disease of poultry, selected from bacterial enteritis, gangrenous dermatitis, colangiohepatitis, clostridiosis, diarrhea and/or foot pad dermatitis, wherein a strain and/or composition and/or preparation of the current invention is administered to an animal in need thereof.

The strains and/or preparations and/or compositions of the current invention can be administered to animals in feed and/or drinking water over multiple days throughout the animal's life or during particular stages or portions of the animal's life. For example, the strains and/or compositions can be administered only in a starter diet or only in a finisher diet of farm animals.

A particular subject of the current invention is also a method of enhancing the health of human beings and/or of improving the general physical condition of human beings and/or of increasing the disease resistance of human beings and/or of increasing the immune response of human beings and/or of establishing or maintaining a healthy gut microflora in human beings, wherein the strains and/or preparations of the current invention or the compositions of the current invention, which comprise such strain(s), are administered to human beings.

A further subject of the current invention is therefore also the use of strains and/or preparations and/or compositions of the current invention for enhancing the health of human beings and/or for improving the general physical condition of human beings and/or for increasing the disease resistance of human beings and/or for increasing the immune response of human beings and/or for establishing or maintaining a healthy gut microflora in human beings, wherein the strains and/or preparations of the current invention or the compositions of the current invention, which comprise such strain(s), are administered to human beings.

The compositions of the present invention, in particular the feed, food and pharmaceutical compositions as well as the drinking or rearing water, preferably comprise the strains of the current invention and are administered to animals at a rate of about 1×10³ to about 2×10¹² CFU/g feed or ml water, in particular in a rate of about 1×10³ or about 1×10⁴ or about 1×10⁵ or about 1×10⁶ or about 1×10⁷ or about 1×10⁸ or about 1×10⁹ or about 1×10¹⁰ or about 1×10¹¹ or about 1×10¹² CFU/g feed or ml water, preferably in an amount of about 1×10⁴ to about 1×10¹⁰ CFU/g feed or ml water, more preferably in an amount of 1×10⁴ to 1×10⁷ CFU/g feed or ml water.

Correspondingly, preferred amounts of the strains and/or preparations of the current invention in the feed, food and water compositions of the current invention range preferably from 0.1 wt.-% to 10 wt.-%, more preferably from 0.2 wt.-% to 5 wt.-%, in particular from 0.3 wt.-% to 3 wt.-%.

The methods of the present invention may be used for all kinds of animals, in particular all kinds of non-human and non-insect animals, more preferably all kinds of vertebrates such as mammals, aquatic animals and birds.

Animals that may benefit from the current invention include but are not limited to farm animals, pets, exotic animals, zoo animals, aquatic animals, animals used for sports, recreation or work.

Pets are preferably selected from dogs, cats, domestic birds and domestic exotic animals. Aquatic animals are preferably selected from finfish and crustaceans which are preferably intended for human nutrition. These include, in particular, carp, tilapia, catfish, tuna, salmon, trout, barramundi, bream, perch, cod, shrimps, lobster, crabs, prawns and crayfish. Preferred types of salmon in this context are the Atlantic salmon, red salmon, masu salmon, king salmon, keta salmon, coho salmon, Danube salmon, Pacific salmon and pink salmon.

Further preferred aquatic animals are farming fish which are subsequently processed to give fish meal or fish oil. In this connection, the fish are preferably herring, pollack, menhaden, anchovies, capelin or cod.

In a further preferred embodiment, the animals are farm animals, which are raised for consumption or as food-producers, such as poultry, swine and ruminants. The poultry may be selected from productive or domestic poultry, but also from fancy poultry or wild fowl.

Preferred productive poultry in this context are chickens, turkeys, ducks and geese. The productive livestock in this context is preferably poultry optimized for producing young stock or poultry optimized for bearing meat.

Preferred fancy poultry or wild fowl are peacocks, pheasants, partridges, chukkars, guinea fowl, quails, capercaillies, grouse, pigeons and swans, with quails being especially preferred.

Further preferred poultry are ratites, in particular ostriches and emus, as well as parrots.

Ruminants according to the current invention are preferably selected from cattle, goat and sheep. In one embodiment, the compositions of this invention may be fed to preruminants to enhance their health and, in particular, to decrease the incidence of diarrhea in these animals. Preruminants are ruminants, including calves, ranging in age from birth to about twelve weeks.

The compositions of the current invention may comprise at least one carrier or typical feed ingredients or combinations thereof.

Suitable carriers are inert formulation ingredients added to improve recovery, efficacy, or physical properties and/or to aid in packaging and administration. Such carriers may be added individually or in combination. These carriers may be selected from anti-caking agents, anti-oxidation agents, bulking agents, and/or protectants. Examples of useful carriers include polysaccharides (in particular starches, maltodextrins, methylcelluloses, gums, chitosan and/or inulins), protein sources (in particular skim-milk powder and/or sweet-whey powder), peptides, sugars (in particular lactose, trehalose, sucrose and/or dextrose), lipids (in particular lecithin, vegetable oils and/or mineral oils), salts (in particular sodium chloride, sodium carbonate, calcium carbonate, chalk, limestone, magnesium carbonate, sodium phosphate, calcium phosphate, magnesium phosphate and/or sodium citrate), and silicates (in particular clays, in particular beolite clay, amorphous silica, fumed/precipitated silicas, zeolites, Fuller's earth, baylith, clintpolite, montmorillonite, diatomaceous earth, talc, bentonites, and/or silicate salts like aluminium, magnesium and/or calcium silicate). Suitable carriers for animal feed additives are set forth in the American Feed Control Officials, Inc.'s Official Publication, which publishes annually. See, for example Official Publication of American Feed Control Officials, Sharon Krebs, editor, 2006 edition, ISBN 1-878341-18-9. The carriers can be added after concentrating the fermentation broth and/or during and/or after drying. Preferred carriers according to the invention are selected from calcium carbonate, diatomaceous earth and vegetable oil.

A preferred embodiment of the current invention are concentrate compositions, in particular feed additive compositions, i.e. compositions suitable for preparing a feed composition, which comprise at least one strain of the current invention and at least one carrier as mentioned before, wherein the at least one strain is preferably comprised in an amount of 0.1 to 10 wt.-%, more preferably in an amount of 0.2 to 5 wt.-%, in particular in an amount of 0.3 to 3 wt.-%, above all in an amount of 0.4 to 2.2 wt.-%, and the at least one carrier is preferably comprised in an amount of at least 90 wt.-%, preferably in an amount of 90 to 99.9 wt.-%, more preferably in an amount of 95 to 99.8 wt.-%, in particular in an amount of 97 to 99.7 wt.-%, above all in an amount of 97.8 to 99.6 wt.-%, and wherein the carrier consists preferably substantially of limestone, in particular of limestone with smaller parts of diatomaceous earth and/or vegetable oil.

These preferred compositions of the current invention, which contain stabilized strains, can be used for the preparation of feed and pharmaceutical compositions as well as drinking and rearing water which preferably comprise the strains according to the invention in an amount as mentioned in the specification above. In a preferred embodiment 200 to 1000 grams of such a concentrate composition, in particular 250, 500 or 1000 grams of such a concentrate composition, are used per ton of feed, drinking or rearing water to provide compositions which can be used for feeding animals. These concentrate compositions preferably comprise at least one strain of the current invention in an amount of 1×10⁹ to 2×10¹¹ CFU, in particular 2×10⁹ to 1×10¹¹ CFU, per g of the concentrate composition.

Starting from these concentrate compositions, feed and food compositions can be prepared by mixing the concentrate compositions with typical feed or food ingredients, respectively.

Suitable typical animal feed ingredients which may be contained in the compositions according to the invention and/or used in the preparation of feed compositions starting from concentrate compositions according to the invention include one or more of the following: proteins, carbohydrates, fats, further probiotics, prebiotics, enzymes, vitamins, immune modulators, milk replacers, minerals, amino acids, coccidiostats, acid-based products and/or medicines, such as antibiotics.

Carbohydrates containing components which may be used according to the invention are for example forage, roughage, wheat meal, sunflower meal or soya meal, and mixtures thereof.

Proteins containing components which may be used according to the invention are for example soya protein, pea protein, wheat gluten or corn gluten, and mixtures thereof.

Fats containing components which may be used according to the invention are in particular oils, of both animal and plant origin, like vegetable oils, for example soya bean oil, rapeseed oil, sunflower seed oil, flaxseed oil or palm oil, fish oil, and mixtures thereof.

Proteins containing components which additionally contain fats which may be used according to the invention are for example fish meal, krill meal, bivalve meal, squid meal or shrimp shells, as well as combinations thereof.

Further probiotics (DFM) which may be used according to the invention in combination with the strains and preparations of the invention are preferably bacteria selected from the species Bacillus subtilis, Bacillus licheniformis, Bacillus lentus, Bacillus pumilus, Bacillus laterosporus, Bacillus coagulans, Bacillus alevi, Bacillus cereus, Bacillus badius, Bacillus thurigiensis, Enterococcus faecium, and Pediococcus acidilactici. Preferred examples are Bacillus subtilis DSM 32315 (as deposited with the DSMZ on May 12, 2016 under the provisions of the Budapest Treaty on the International Recognition of the Deposit of Microorganisms for the Purpose of Patent Procedure) and derivatives thereof, Bacillus subtilis PB6 (as described in U.S. Pat. No. 7,247,299 and deposited as ATCC Accession No. PTA-6737), which is sold by Kemin under the trademark CLOSTAT®, Bacillus subtilis C-3102 (as described in U.S. Pat. No. 4,919,936 and deposited as FERM BP-1096 with the Fermentation Research Institute, Agency of Industrial Science and Technology, in Japan), sold by Calpis as CALSPORIN®, Bacillus subtilis DSM 17299, as sold by Chr. Hansen under the trademark GalliPro®, a mixture of Bacillus subtilis DSM 17299 and Bacillus licheniformis DSM 17236, as sold by Chr. Hansen under the trademark GalliProTect®, a mixture of Bacillus licheniformis and Bacillus subtilis spores sold by Chr. Hansen under the trademark BIOPLUS2B®, or Bacillus coagulans strains as described in U.S. Pat. No. 6,849,256. Other non-Bacillus probiotics, such as Saccharomyces cerevisiae, Pichia pastoris, Aspergillus niger, Aspergillus oryzae, or Hansenula, may also be used in compositions of the present invention. In particular in food compositions further probiotics which are known to be useful to the human health may be used such as lactic acid producing bacteria, in particular lactobacilli, or Bifidobacteria. If said further probiotics are not formulated as part of the compositions of the present invention, they may be administered together (either at the same time or at different times) with the compositions of the present invention.

Prebiotics which may be used according to the invention are preferably oligosaccharides, in particular selected from galactooligosaccharides, silayloligosaccharides, lactulose, lactosucrose, fructooligosaccharides, palatinose or isomaltose oligosaccharides, glycosyl sucrose, maltooligosaccharides, isomaltooligosaccharides, cyclodextrins, gentiooligosaccharides, soybean oligosaccharides, xylooligosaccharides, dextrans, pectins, polygalacturonan, rhamnogalacturonan, mannan, hemicellulose, arabinogalactan, arabinan, arabinoxylan, resistant starch, mehbiose, chitosan, agarose, inulin, tagatose, polydextrose, and alginate.

Enzymes which may be used in feed compositions according to the invention and which may aid in the digestion of feed, are preferably selected from phytases (EC 3.1 .3.8 or 3.1.3.26), xylanases (EC 3.2.1.8), galactanases (EC 3.2.1 .89), galactosidases, in particular alpha-galactosidases (EC 3.2.1.22), proteases (EC 3.4), phospholipases, in particular phospholipases A1 (EC 3.1 .1.32), A2 (EC 3.1.1.4), C (EC 3.1.4.3), and D (EC 3.1.4.4), lysophospholipases (EC 3.1 .1.5), amylases, in particular alpha-amylases (EC 3.2.1.1); lysozymes (EC 3.2.1 .17), glucanases, in particular beta-glucanases (EC 3.2.1.4 or EC 3.2.1.6), glucoamylases, cellulases, pectinases, or any mixture thereof.

Examples of commercially available phytases include Bio-Feed™ Phytase (Novozymes), Ronozyme® P and HiPhos™ (DSM Nutritional Products), Natuphos™ (BASF), Finase® and Quantum® Blue (AB Enzymes), the Phyzyme® XP (Verenium/DuPont) and Axtra® PHY (DuPont). Other preferred phytases include those described in e.g. WO 98/28408, WO 00/43503, and WO 03/066847.

Examples of commercially available xylanases include Ronozyme® WX and G2 (DSM Nutritional Products), Econase® XT and Barley (AB Vista), Xylathin® (Verenium) and Axtra® XB (Xylanase/beta-glucanase, DuPont). Examples of commercially available proteases include Ronozyme® ProAct (DSM Nutritional Products).

Vitamins which may be used according to the invention are for example vitamin A, vitamin D3, vitamin E, vitamin K, e.g., vitamin K3, vitamin B12, biotin, choline, vitamin B1 , vitamin B2, vitamin B6, niacin, folic acid and panthothenate, e.g. , Ca-D-panthothenate, or combinations thereof.

Immmune modulators which may be used are for example antibodies, cytokines, spray-dried plasma, interleukins, or interferons, or combinations thereof.

Minerals which may be used according to the invention are for example boron, cobalt, chloride, chromium, copper, fluoride, iodine, iron, manganese, molybdenum, selenium, zinc, calcium, magnesium, potassium, or sodium, or combinations thereof.

Amino acids which may be used according to the invention are for example lysine, alanine, threonine, methionine or tryptophan, or combinations thereof.

Thus, a further embodiment of the current invention is a method of preparing an animal feed composition comprising mixing at least one strain and/or at least one preparation and/or at least one concentrate composition of the current invention, in particular in an amount effective to enhance animal health, with feed ingredients, such as proteins, lipids and/or carbohydrates, and optionally further beneficial substances, preferably as mentioned before, to provide a feeding product. This method may comprise for example also a pelleting step.

Standard pelleting processes known to those of skill in the art may be used, including extrusion processing of dry or semi-moist feeds. Preferred pelleting temperatures are between about 65° C. and about 120° C.

The strains and compositions of the present invention can be obtained by culturing the strains of the current invention according to methods well known in the art, including by using the media and other methods as described for example in U.S. Pat. No. 6,060,051, EP0287699 or US2014/0010792. Conventional large-scale microbial culture processes include submerged fermentation, solid state fermentation, or liquid surface culture. Towards the end of fermentation, as nutrients are depleted, the cells of the strains begin the transition from growth phase to sporulation phase, such that the final product of fermentation is largely spores, metabolites and residual fermentation medium. Sporulation is part of the natural life cycle of these strains and is generally initiated by the cell in response to nutrient limitation. Fermentation is configured to obtain high levels of colony forming units of the Bacillus licheniformis cells and to promote sporulation. The bacterial cells, spores and metabolites in culture media resulting from fermentation may be used directly or concentrated by conventional industrial methods, such as centrifugation, tangential-flow filtration, depth filtration, and evaporation. The concentrated fermentation broth may be washed, for example via a diafiltration process, to remove residual fermentation broth and metabolites.

The fermentation broth or broth concentrate can be dried with or without the addition of carriers using conventional drying processes or methods such as spray drying, freeze drying, tray drying, fluidized-bed drying, drum drying, or evaporation. The resulting dry products may be further processed, such as by milling or granulation, to achieve a specific particle size or physical format. Carriers, as described above, may also be added post-drying.

Preparations of the strains of the current invention may be cell-free preparations or preparations containing cell debris or preparations containing a mixture of intact cells and cell debris. Cell-free preparations of the strains of the current invention can be obtained for example by centrifugation and/or filtration of fermentation broth. Depending on the technique used, these cell-free preparations may not be completely devoid of cells, but may still comprise a smaller amount of cells. As the cells secret compounds like metabolites, enzymes and/or peptides into the surrounding medium, the supernatant of the cells comprises a mixture of such compounds, in particular metabolites, enzymes and/or peptides, as secreted by the cells. Thus, in a preferred embodiment of the invention, the preparation of the strains is a supernatant of the fermentation broth.

Compositions comprising cell debris of the strains may be obtained by rupturing the cells applying techniques as known to those of skill in the art, for example by mechanical means or by applying high pressure. Depending on the degree of force applied, a composition comprising only ruptured cells or a composition comprising a mixture of cell debris and intact cells is obtained. Homogenization of the cells may be realized for example by utilizing a French cell press, sonicator, homogenizer, microfluidizer, ball mill, rod mill, pebble mill, bead mill, high pressure grinding roll, vertical shaft impactor, industrial blender, high shear mixer, paddle mixer, and/or polytron homogenizer. Suitable alternatives are enzymatic and/or chemical treatment of the cells.

Cell-free preparations of the current invention comprise also preparations which are obtained by first rupturing the cells by applying techniques as mentioned before and subsequently removing the cell debris and the remaining intact cells. Removing of the cell debris and remaining intact cells can be carried out in particular by centrifugation and/or filtration.

The preparations of the strains of the current invention may comprise as active compounds at least one metabolite, preferably a mixture of metabolites, as further described below, and/or at least one enzyme selected from proteases, in particular lichenisin, xylanases and/or cellulases, and/or at least one peptide, and/or combinations thereof.

A preparation containing an effective mixture of metabolites as contained in the strains of the current invention and/or as contained in the cell preparations as mentioned before, can be obtained for example according to the methods set forth in U.S. Pat. No. 6,060,051. In particular the preparation can be obtained by precipitating the metabolites as contained in the preparations mentioned before by using organic solvents like ethyl acetate and subsequent redissolving of the precipitated metabolites in an appropriate solvent. The metabolites may subsequently be purified by size exclusion filtration that groups metabolites into different fractions based on molecular weight cut-off.

The preparation containing an effective mixture of metabolites of the current invention preferably comprises at least five, more preferably at least 6, 7, 8 or 9, in particular all metabolites of the strains of the invention. The content of metabolites of the strain DSM 32314 is depicted in Table 5.1. The metabolites possess preferably a molecular weight of between 400 and 3000 Dalton, more preferably of between 450 and 2500 Dalton.

Preferably according to the invention always an effective amount of the strains and/or preparations and/or compositions of the current invention is used in the embodiments of the current invention. The term “effective amount” refers to an amount which effects at least one beneficial effect to an animal and/or to the environment, in particular with respect to the features as already mentioned before, in comparison to an animal that has not been administered the strains and/or preparations and/or compositions of the current invention, but besides that has been administered the same diet (including feed and other compounds).

In case of therapeutic applications preferably a therapeutic amount of the strains and/or preparations and/or compositions of the current invention is used. The term “therapeutic amount” refers to an amount sufficient to ameliorate, reverse or prevent a disease state in an animal. Optimal dosage levels for various animals can easily be determined by those skilled in the art, by evaluating, among other things, the composition's ability to (i) inhibit or reduce pathogenic bacteria in the gut at various doses, (ii) increase or maintain levels of beneficial bacteria and/or (iii) enhance animal health at various doses.

### WORKING EXAMPLES

### Example 1. Strain Characteristics Relevant to Survival in the Gastrointestinal Tract

Bacillus licheniformis strains were screened from various environmental samples in order to obtain a superior strain as animal direct-fed microbial (DFM)/probiotic. As the strain is intended to reach its full potential in the intestine of the target animal, the strain was pre-screened to withstand various environmental and gut related conditions. Strain spores were generated (Nicholson and Setlow 1990), washed and incubated at 80° C. for 20 minutes (pasteurization), then titrated in logarithmic/1 in 10 dilutions using veal infusion broth agar (VI, Difco™, no. 234420, Becton Dickinson GmbH, Heidelberg, Germany). The second highest dilution prior to no growth was stored at −80° C. and used as standardized starting point for all further assessments from spore state. To simulate gastric passage (Argenzio 2004a; Trampel and Duke 2004), survival of acid exposure was assessed based on Larsen et al. (2014). Growth of vegetative cells was furthermore assessed at low pH indicating growth under stomach/proventriculus and gizzard conditions, as well as in presence of up to 4 mM bile (B8631, CAS 8008-63-8, Sigma-Aldrich) at pH 7 in order to confirm strain growth at the proximal part of the small intestine right after clearance of the stomach or gizzard (Argenzio 2004b; Trampel and Duke 2004).) Strain fitness in the anaerobe intestine (Argenzio 2004b; Trampel and Duke 2004) was assessed by inoculating standardized spore solutions under anaerobic conditions (AnaeroPak™, Thermo Fisher Scientific) in VI medium supplemented with 2.5 mM KNO₃ (Glaser et al. 1995). Furthermore was the anaerobe proteolytic and cellulytic activity of strains assessed on VI agar plates supplemented with 1% skim milk powder (70166, Sigma-Aldrich) or 0.1% water insoluble AZCL-HE cellulose (I-AZCEL, Megazyme International, Bray, Ireland). Osmotic stress, as also found in the gut (Argenzio 2004b; Trampel and Duke 2004), was assessed by determining growth on VI agar with addition of 5% NaCl (den Besten et al. 2009). Finally, spore heat stability was assessed to determine pelleting stability by exposing spores to 99° C. for 20 min (Palop et al. 1996) and subsequent inoculation on VI agar. Bacillus licheniformis strain DSM 32314 survived simulated gastric passage, growth of strain DSM 32314 was observed starting at pH 6. Strain DSM 32314 grew anaerobically and was able to degrade water-insoluble cellulose under anaerobic conditions. Strain DSM 32314 was able to grow in presence of 2 and 4 mM bile, as well as in presence of 10% NaCl. Strain DSM 32314 reached an average spore count of 4.35×10⁹ CFU/mL, and spores of strain DSM 32314 were viable after exposure to 99° C. for 20 min. Furthermore, B. licheniformis wildtype strains WT1 and WT2 were assessed to grow anaerobically and to withstand pH 6, but they were unable to anaerobically degrade cellulose and were therefore disqualified as DFM/probiotic candidates. However, they were used as wildtype comparison in the following examples.

### REFERENCES

Argenzio, R. A. (2004a). Secretion of the Stomach and Accessory Glands, p. 405-418. In: Reece, W. O. (ed.), Duke's Physiology of Domestic Animals; Twelfth Edition, Chapter 25; Cornell University Press, Ithaca, N.Y., USA.

Argenzio, R. A. (2004b). Digestive and Absorptive Functions of the Intestines, p. 419-437. In: Reece, W. O. (ed.), Duke's Physiology of Domestic Animals; Twelfth Edition, Chapter 26; Cornell University Press, Ithaca, N.Y., USA.

Dawson, R. M. C.; Elliot, D. C.; Elliot, W. H.; Jones, K. M. (1986). Data for Biochemical Research; 3^{rd} edition, Oxford Science Publishing, United Kingdom.

Den Besten HMW, Mols M, Moezelaar R, Zwietering MH, Abee T. (2009). Phenotypic and transcriptomic analyses of mildly and severely salt-stressed Bacillus cereus ATCC 14579 cells. Appl Environ Microbiol. 75:4111-9.

Glaser, P., A. Danchin, F. Kunst, P. Zuber, and M. M. Nakano. (1995). Identification and isolation of a gene required for nitrate assimilation and anaerobic growth of Bacillus subtilis. J. Bacteriol. 177:1112-1115

Nicholson W. L., Setlow P. Sporulation, germination and outgrowth. In: Harwood C R, Cutting S M, editors. Molecular biological methods for Bacillus. Chichester, England: John Wiley & Sons Ltd.; 1990. pp. 27-74.

Palop, A., Raso, J., Pagan, R., Condon, S. and Sala, F. J. (1996). Influence of pH on heat resistance of Bacillus licheniformis in buffer and homogenized foods. International Journal of Food Microbiology 29, 1-10.

Trampel, D. W. and Duke, G. E. (2004). Avian Digestion, p. 488-500. In: Reece, W. O. (ed.), Duke's Physiology of Domestic Animals; Twelfth Edition, Chapter 29; Cornell University Press, Ithaca, N.Y., USA.

### Example 2. Comparative Strain Performance Relative to Wild-Type Bacillus licheniformis—Quantitative Assessment of Bile Tolerance

In order to assess the competitiveness of the Bacillus licheniformis strain DSM 32314 selected from example 1, analysis was performed comparing to representative Bacillus licheniformis wildtype 1 (WT1) and wildtype 2 (WT2) and readiness of strains to perform in the proximal small intestine in presence of bile at neutral pH right after gastric passage (Argenzio 2004b; Trampel and Duke 2004) was determined by strain growth in VIB media with addition 2 mM bile. Overnight culture with 50 uL candidate strain cell suspension and 10 mL VIB in 100 mL conical flask was incubated at 37° C. and 200 rpm, then approximately 50 uL of overnight culture was transferred to 100 well honeycomb plates (Oy Growth Curves Ab Ltd, former Thermo Labsystems, Helsinky, Finland) with 1 mL VIB at pH 7 with 2 mM bile in order to obtain OD 0.2 per mL. Strain specific growth at 37° C. and 200 rpm was observed for 48 h with OD determined every 15 mM using Bioscreeen C MBR with BioLink software package (Oy Growth Curves Ab Ltd). Averaged triplicate blank OD read of broth with bile only (blanks) were subtracted per culture at each time point before area under the curve (AUC) was calculated. Quantitative assessment for each strain was compared as area under the curve between 0-5 h (AUC5, in OD×time in h), area under the curve between 0-10 h (AUC10 in OD×time in h), and time until strains reached its maximum optical density (Tmax in h). Statistical analysis was performed using one-way ANOVA procedure of MiniTab ® 16 Statistical Software (Minitab Inc., State College, Pa., USA). Results can be found in Table 2.1.

TABLE 2.1
Growth of Bacillus licheniformis strains DSM 32314 and
wildtype strains WT1 and WT2 in presence of 2 mM bile.

| Strain ID | AUC5 | AUC10 | Tmax |
|---|---|---|---|
| DSM 32314 | 0.539 ^{A} | 2.144 ^{A} | 18.2 ^{C} |
| WT1 | 0.266 ^{B} | 0.714 ^{B} | 30.3 ^{B} |
| WT2 | 0.378 ^{B} | 1.177 ^{B} | 36.4 ^{A} |
| P-value | P < 0.01 | P = 0.001 | P < 0.001 |
| SEM | 0.018 | 0.076 | 0.5 |

AUC5, area under the curve between time point 0 and 5 h in optical density×h; AUC10, area under the curve between time point 0 and 10 h in optical density×h; Tmax, time in h until maximum optical density was reached; SEM, pooled standard error of the mean;^{A, B} means that do not share a letter are significantly different.

In direct comparison, strain DSM 32314 reached its maximum OD in presence of 2 mM bile 10 h faster than the wildtype Bacillus licheniformis strains WT1 and WT2. In addition, strain DSM 32314 grew 1.4-2.0 fold faster during the first 5 hours, and 1.8-3.0 fold faster during the first 10 h of the test, compared to growth of wildtype B. licheniformis strains, respectively.

### REFERENCES

Argenzio, R. A. (2004b). Digestive and Absorptive Functions of the Intestines, p. 419-437. In: Reece, W. O. (ed.), Duke's Physiology of Domestic Animals; Twelfth Edition, Chapter 26; Cornell University Press, Ithaca, N.Y., USA.

Trampel, D. W. and Duke, G. E. (2004). Avian Digestion, p. 488-500. In: Reece, W. O. (ed.), Duke's Physiology of Domestic Animals; Twelfth Edition, Chapter 29; Cornell University Press, Ithaca, N.Y., USA.

### Example 3. Comparative Strain Performance Relative to State of the Art Direct-Fed Microbial (DFM)/Probiotic for Animal Nutrition—Growth in Presence of Short Chain Fatty Acids (SCFA)

Comparative growth of strains DSM 32314 and WT1 was assessed in presence of short chain fatty acids as those are observed in the gut with increasing importance towards the large intestine (Argenzio 2004b; Trampel and Duke 2004). Tests were initiated using standardized spore solution as described in example 1 testing aerobe growth in VI medium at 37° C. and pH 6, read-out parameter was growth versus no growth. For this test, VI medium was adjusted to pH 6 using Mcllvaine buffer (Palop et al. 1996) and subsequently supplemented with 0.05% acetic acid (HA, 537020, CAS 64-19-7, Sigma-Aldrich), 0.05% propionic acid (HP, P1386, CAS 79-09-4, Sigma-Aldrich) or 0.2% lactic acid (HL, W261106, CAS 50-21-5, Sigma-Aldrich). Results can be found in Table 3.1.

TABLE 3.1
Assessment of growth of Bacillus licheniformis
strain DSM 32314 and wildtype strain WT1 in presence
of short chain fatty acids at pH 6.

| Strain ID | Acetic acid | Propionic acid | Lactic acid |
|---|---|---|---|
| DSM 32314 | Yes | Yes | Yes |
| WT1 | No growth | No growth | No growth |

Bacillus licheniformis strain DSM 32314 was able to grow at pH 6 in the presence of acetic, propionic and lactic acid, whereas WT1 strain was unable to grow from spore stage under these conditions.

### REFERENCES

Argenzio, R. A. (2004b). Digestive and Absorptive Functions of the Intestines, p. 419-437. In: Reece, W. O. (ed.), Duke's Physiology of Domestic Animals; Twelfth Edition, Chapter 26; Cornell University Press, Ithaca, N.Y., USA.

Palop, A., Raso, J., Pagan, R., Condon, S. and Sala, F.J. (1996). Influence of pH on heat resistance of Bacillus licheniformis in buffer and homogenized foods. International Journal of Food Microbiology 29, 1-10.

Trampel, D. W. and Duke, G. E. (2004). Avian Digestion, p. 488-500. In: Reece, W. O. (ed.), Duke's Physiology of Domestic Animals; Twelfth Edition, Chapter 29; Cornell University Press, Ithaca, N.Y., USA.

### Example 4. Comparative Strain Performance Relative to State of the Art Direct-Fed Microbial (DFM)/Probiotic for Animal Nutrition—Quantitative Assessment of Enzymatic Activity

Similar to test conducted in Example 2, strains DSM 32314, WT1 and WT2 were compared evaluating the respective aerobe cellulytic, xylanolytic and proteolytic activity. Cellulase and Xylanase activity were determined as described in Larsen et al. (2014). For proteolytic activity analysis, starter and main culture of strains were grown in VIB at 37° C. as previously described. From main culture, 10 uL were added to 20 uL 0.5% Fluorescein Isothiocyanate Casein (FITC; C3777, Sigma-Aldrich) solution with 20 uL buffer consisting of 20mM sodium phospate (dibasic, anhydrous) with 150 mM sodium chloride (all components from Sigma-Aldrich), then incubated for 1 h at 37° C. After addition of 150 uL of 10% (v/v) trichlor acetic acid (Sigma-Aldrich) and another 30 min incubation at 37° C., samples were centrifuged at 19,000 rpm for 15 min, then 2 uL of supernatant transferred to 200 uL 500 mM TRIS HCl Solution (Trizma BaseTRIS, Sigma-Aldrich). Fluorescence of soluble peptides due to proteolytic release were determined (TECAN GENios Microplate Reader, Tecan Group Ltd., Männedorf, Switzerland) at excitation 494 nm, emission 518 nm. Analysis was performed in three independent runs, then averaged as milliunits per microliter solution, statistical analysis was performed using one-way ANOVA procedure of MiniTab ® 16 Statistical Software (Minitab Inc., State College, Pa., USA). Results can be found in Table 4.1.

TABLE 4.1
Cellulase, protease and xylanase activity of Bacillus licheniformis
strain DSM 32314 compared to wildtype strains WT1 and WT2.

| | Cellulase activity | Protease activity | Xylanase activity |
|---|---|---|---|
| Strain ID | (mU/mL) | (mU/mL) | (mU/mL) |
| DSM 32314 | 250.3 A | 9.8 A | 20.5 A |
| WT1 | n/d | 3.3 B | 14.3 B |
| WT2 | 56.6 B | 2.7 B | 14.6 B |
| P-value | P < 0.001 | P < 0.005 | P < 0.001 |
| SEM | 15.3 | 1.8 | 0.8 |

SEM, pooled standard error of the mean;
A, B means that do not share a letter are significantly different.
In direct comparison, strain DSM 32314 demonstrated significant 4.4 fold increased cellulase activity comparing to WT2. In addition, DSM 32314 demonstrated significant 3.0 fold increased protease activity comparing to WT1 and 3.6 fold increased protease activity comparing to WT2. Moreover, DSM 32314 demonstrated significant 1.4 fold increased xylanase activity comparing to WT1 and WT2.

### REFERENCE

Larsen, N., Thorsen, L., Kpikpi, E. N., Stuer-Lauridsen, B., Cantor, M. D., Nielsen, B., Brockmann, E., Derkx, E. M. F. and Jespersen, L. (2014). Characterization of Bacillus spp. strains for use as probiotic additives in pig feed. Applied microbiology and biotechnology, 98(3), 1105-1118.

### Example 5. Comparative Strain Performance Relative to State of the Art Direct-Fed Microbial (DFM)/Probiotic for Animal Nutrition—Expression of Metabolites and Pathogen Inhibition

Similar to tests conducted in Example 2, B. licheniformis strains DSM 32314, WT1 and WT2 were compared evaluating the respective number of metabolites expressed and pathogens inhibited in the respective media. For metabolite expression analysis, starter cultures were grown and tests performed as described in Scholz et al. (2011). From 10 mL Luria Bertami broth (LB, Thermo Fisher Scientific) culture grown for 24 h at 37° C. and 160 rpm in 100 mL flask, 100 uL were transferred to main culture. Main culture was grown either in 10 mL LB containing 0.2 mL/L KellyT trace metal solution (LBKelly, Scholz et al. 2011), or 10 mL Trypticase Soy Broth (Oxoid, Thermo Fisher Scientific) with 0.6% yeast extract (Y1625, CAS 8013-1-2, Sigma-Aldrich; resulting broth abbreviated TSBYE), both for 24 hat 37° C. at 160 rpm in 100 mL flask. Of the main culture, 4 mL were combined with 2 mL n-Butanol in 15 mL test tube, vortexed for 3 min, then sonicated for 15 min. After centrifugation for 1 min at 5000 rpm, organic phase was transferred, vacuum dried and analyzed using High-performance liquid chromatography—electrospray ionization mass spectrometry (HPLC-ESI-MS; Chen et al. 2006). Every sample was measured in two different modes, negative and positive mode, and mass spectra were acquired. Resulting peaks as similarly reported in Teo and Tan (2005) were converted to molecular mass in Da. Results for comparison can be found in Table 5.1.

TABLE 5.1
Comparison of metabolites expressed by strains DSM
32314, WT1 and WT2, in L B Kelly, respectively.

| | Molecular Mass | | | | | | | | | | | | |
|---|---|---|---|---|---|---|---|---|---|---|---|---|---|
| | 496 | 389 | 855 | 865 | 943 | 969 | 1008 | 1022 | 1036 | 1050 | 1420 | 1423 | 2471 |
| | Da | Da | Da | Da | Da | Da | Da | Da | Da | Da | Da | Da | Da |
| DSM 32143 | yes | n/d | yes | n/d | n/d | yes | yes | yes | yes | yes | yes | yes | yes |
| WT1 | n/d | yes | n/d | yes | yes | n/d | n/d | n/d | n/d | n/d | n/d | n/d | n/d |
| WT2 | n/d | n/d | n/d | n/d | n/d | n/d | yes | yes | traces | n/d | n/d | n/d | n/d |

In addition, Clostridium perfringens inhibition via Bacillus licheniformis bacteriocin production, as part of metabolites from table 5.1. but not closer investigated, was assessed using well diffusion antagonismus test (Parente et al. 1995). Four pathogenic C. perfringens candidates were tested being C. perfringens type strain ATCC 13124 from Teo and Tan (2005), as well as three pathogenic C. perfringens field isolates from poultry and swine, obtained from University of Leipzig, Faculty of Veterinary Medicine, Department of Bacteriology and Mycology, Prof. Dr. Christoph Baums, Potsdam, Germany. The C. perfringens type A-strains from Leipzig describe as follows: Strains 2300-1-17 and 2300-1-18 were isolated from necrotic enteritis positive chicken digestive tract. Both strains produce α-toxin, strain 2300-1-17 also expressing NetB toxin (Savva et al. 2013; Uzal et al. 2014,) whereas strain 2300-1-18 tested positive for β2-toxin (Allaart et al. 2012). Strain 2300-1-19 was isolated from digestive tract of a scouring piglet exhibiting symptoms of Clostridial type A enteritis (Songer and Uzal 2005). Growth conditions and media were described by Teo and Tan (2005). In brief, Bacillus strains were grown in 10 mL TSBYE and LBKelly starter culture for 24 hat 37° C. and 160 rpm in 100 mL flask in 5% CO₂ atmosphere, respectively. Clostridium perfringens starter cultures were cultivated anaerobically (AnaeroPak™, Thermo Fischer Scientific) in fluid thioglycolate broth (FTB, Becton Dickenson) for 24 h at 37° C. and 160 rpm in 100 mL flask, then spread with sterile cotton swab agar plates (TSBYE with 1% agar, short TSAYE). Inoculated Inoculated TSAYE plates were then incubated anaerobically overnight at 37° C. in order to obtain lawn of C. perfringens. After overnight growth, three 9 mm diameter wells were cut into the agar with lawn, 1S^{t} well was used as non-inoculated control without culture, 2^{nd} well was inoculated with 100 uL not—C. perfringens-inhibiting Bacillus strain (B. cereus var. toyoi, NCIMB 40112), the 3^{rd} well was inoculated with 100 uL of Bacillus licheniformis DSM 32314 or DSM 17299 starter culture. After 24 h incubation at 37° C., zone of clearance in mm was determined measuring from the edge of the cut well to the border of the cleared lawn, each colony was measured twice (horizontally, vertically), then averaged. For each Bacillus licheniformis antagonism test and media, analysis was performed in duplicate plate runs. Statistical analysis was performed using one-way ANOVA procedure of MiniTab ® 16 Statistical Software (Minitab Inc., State College, Pa., USA) using Fisher LSD for mean separation, results can be found Table 5.2. for pathogen inhibition (strains grown in LBKelly), in Table 5.3 for pathogen inhibition (strains grown in TSBYE).

TABLE 5.2
Comparison of Bacillus licheniformis DSM 32314
and DSM 17299 inhibitory capacity of pathogenic C. perfringens
well diffusion assay (strains grown in LBKelly),
values in mm clearance of pathogen.

| | Pathogenic C. perfringens | | | | |
|---|---|---|---|---|---|
| | Poultry | Poultry | Swine | | |
| | Necrotic | Necrotic | Clostridial | Type | |
| | Enteritis | Enteritis | Enteritis | strain | |
| | strain | strain | strain | ATCC | Average |
| Bacillus | 2300-1-17 | 2300-1-18 | 2300-1-19 | 13124 | inhibition |
| DSM 32314 | 15.50 | 11.50 | 14.50 | 14.00 | 13.88 ^{A} |
| DSM 17299 | 2.00 | 0.00 | 6.00 | 2.00 | 2.50 ^{B} |
| P-value | | | | | <0.001 |
| SEM | | | | | 2.15 |

SEM, pooled standard error of the mean;
^{A, B} means that do not share a letter are significantly different.

TABLE 5.3
Comparison of Bacillus licheniformis DSM 32314 and
DSM 17299 inhibitory capacity of pathogenic C. perfringens
well diffusion antagonism (strains grown in TSBYE),
values in mm clearance of pathogen.

| | Pathogenic C. perfringens | | | | |
|---|---|---|---|---|---|
| | Poultry | Poultry | Swine | | |
| | Necrotic | Necrotic | Clostridial | Type | |
| | Enteritis | Enteritis | Enteritis | strain | |
| Bacillus | strain | strain | strain | ATCC | Average |
| strains | 2300-1-17 | 2300-1-18 | 2300-1-19 | 13124 | inhibition |
| DSM 32314 | 21.00 | 17.00 | 21.00 | 18.00 | 19.25 ^{A} |
| DSM 17299 | 0.00 | 0.00 | 0.00 | 0.00 | 0.00 ^{B} |
| P-value | | | | | <0.001 |
| SEM | | | | | 1.46 |

SEM, pooled standard error of the mean;
^{A, B} means that do not share a letter are significantly different.

### REFERENCES

Allaart, J. G., de Bruijn, N. D., van Asten, A. J., Fabri, T. H., and Gröne, A. (2012). NetB-producing and beta2-producing Clostridium perfringens associated with subclinical necrotic enteritis in laying hens in the Netherlands. Avian Pathol., 41:541-546

Chen, X. H., Vater, J., Piel, J., Franke, P., Scholz, R., Schneider, K., Koumoutsi, A., Hitzeroth, G., Grammel, N., Strittmatter, A. W., Gottschalk, G., Süssmuth, R. and Borriss, R. (2006). Structural and functional characterization of three polyketide synthase gene clusters in Bacillus amyloliquefaciens FZB 42. Journal of bacteriology, 188(11), 4024-4036.

Parente, E., Brienza, C., Moles, M., & Ricciardi, A. (1995). A comparison of methods for the measurement of bacteriocin activity. Journal of microbiological methods, 22(1), 95-108.

Sawa, G. S., Fernandes da Costa, S. P., Bokori-Brown, M., Naylor, C. E., Cole, A. R., Moss, D. S., Titball, R-W., and Basak, A. 2013. Molecular architecture and functional analysis of NetB, a pore-forming toxin from Clostridium perfringens. J Biol. Chem., 288: 3512-3522.

Scholz, R., Molohon, K. J., Nachtigall, J., Vater, J., Markley, A. L., Süssmuth, R. D., Mitchell, D. A. and Borriss, R. (2011). Plantazolicin, a novel microcin B17/streptolysin S-like natural product from Bacillus amyloliquefaciens FZB42. Journal of bacteriology, 193(1), 215-224.

Songer, J. G., and Uzal, F. A. (2005). Clostridial enteric infections in pigs. Journal of veterinary diagnostic investigation, 17(6), 528-536.

Teo, A. Y.-L. and Tan, H.-M. (2005). Inhibition of Clostridium perfringens by a novel strain of Bacillus licheniformis from the gastrointestinal tracts of healthy chickens. Appl. Environm. Microbiol., 71:4185-90.

Uzal, F. A., Freedman, J. C., Shrestha, A., Theoret, J. R., Garcia J., Awad, M. M., Adams, V., Moore, R. J., Rood, J. I., and McClane, B. A. (2014). Towards an understanding of the role of Clostridium perfringens toxins in human and animal disease. Future Microbiol., 9:361-377

### Example 6. Comparison of Performance of Broilers Reared in India Fed a Novel Bacillus licheniformis or an Antibiotic Growth Promoter

A broiler growth performance study was conducted with day-old, male, Vencobb 400 (Venkateshwara Hatcheries Pvt. Ltd, India) chicks placed in floor pens with used litter. Three dietary treatments were randomly assigned, with 18 replicate pens per treatment containing 25 birds per pen. Birds were fed with one of the dietary treatments in three phases consisting of starter (1-14 days), grower (15-28 days) and finisher (29-42 days) phases. The basal diet was mainly based on corn-soybean meal (Table 6.1) containing 500g/MT of dinotolmide to control coccidiosis. The basal diet also included 4% meat and bone meal (MBM), as additional challenge as MBM is a predisposing factor for development of necrotic enteritis caused by Clostridium perfringens in broiler chickens (M'Sadeqa et al. 2015). Three dietary treatments were mainly based on corn-soybean meal (Table 6.1) and included; 1. Basal control (Control), 2. Control+50 g of Bacitracin Methylene Disalicyclate/MT of feed (BMD), 3. Control+a Bacillus licheniforomis strain DSM 32314 at 250 g/MT containing 2.0*109 cfu/g (DSM 32314). Experimental treatments were fed ad libitum in mash form from 1-42 days of age. Statistical analysis was performed using one-way ANOVA procedure and LSD post-test analysis with SAS vs9.4 (SAS Institute Inc., USA). The results of the treatments on body weight, feed conversion ratio and mortality are reported in Table 6.2.

TABLE 6.1
Basal diet and diet nutrient composition
for starter grower and finisher phases.

| | Starter | Grower | Finisher |
|---|---|---|---|
| | (1-14 d) | (15-28 d) | (29-42 d) |
| Ingredients, % | | | |
| Corn | 53.98 | 62.27 | 64.54 |
| Soybean meal, 48% CP | 36.09 | 27.64 | 24.63 |
| Meat and bone meal | 4.00 | 4.00 | 4.00 |
| Soybean oil | 2.46 | 2.60 | 3.60 |
| Dicalcium phoslphate | 1.48 | 1.51 | 1.53 |
| Limestone | 0.39 | 0.45 | 0.16 |
| Premix | 0.65 | 0.65 | 0.65 |
| (including vit-min mix) | | | |
| Sodium chloride | 0.28 | 0.29 | 0.29 |
| Sodium bicarbonate | 0.10 | 0.10 | 0.10 |
| Choline chloride 50 | 0.10 | 0.10 | 0.10 |
| DL-Methionine | 0.29 | 0.23 | 0.22 |
| L-Lysine HCl | 0.12 | 0.13 | 0.14 |
| L-Threonine | 0.05 | 0.04 | 0.05 |
| Nutrient composition | | | |
| ME, kcal/kg | 2950 | 3050 | 3150 |
| CP, % | 23.50 | 20.08 | 18.83 |
| Ca | 1.00 | 1.00 | 1.00 |
| Available P | 0.45 | 0.45 | 0.45 |
| Lys | 1.36 | 1.14 | 1.06 |
| Met | 0.63 | 0.53 | 0.50 |
| M + C | 0.99 | 0.85 | 0.80 |
| Thr | 0.92 | 0.78 | 0.74 |
| Trp | 0.27 | 0.22 | 0.20 |
| Arg | 1.59 | 1.33 | 1.23 |
| Ile | 0.97 | 0.81 | 0.75 |
| Leu | 1.93 | 1.70 | 1.61 |
| Val | 1.08 | 0.92 | 0.86 |

TABLE 6.2
Animal performance between days 0 and 42 with and without
diet supplementation with B. licheniformis based
feed additive or antibiotic growth promoter.

| | |
|---|---|
| 1-21 d | 1-42 d |

| Treatment | BW, g | FCR, g/g | BW, g | FCR, g/g |
|---|---|---|---|---|
| 1. Control | 880.7 | 1.43 | 2757.4 | 1.76 |
| 2. BMD | 899.0 | 1.42 | 2781.6 | 1.74 |
| 3. DSM 32314 | 905.2 | 1.42 | 2824.2 | 1.74 |
| Difference | 24.5 | −0.01 | 66.8 | −0.02 |
| Relative % | 2.8 | −0.70 | 2.4 | −1.14 |

BW, average bird body weight in specified time period; FCR, feed conversion ratio calculated as feed to gain in specified time period; Control, no additives in basal diet; BMD, treatment with addition of bacitracin methylene disalicylate to basal diet; DSM 32314, treatment with addition of strain DSM 32314 to basal diet; Difference, the numeric difference observed when DSM 32314was compared to control; Relative %, the difference between DSM 32314and control as a percent change from control.

The data from this broiler indicates that both products, BMD and DSM 32314, improved the body weight compared to the control. However, the pre-product group D treated group had the highest average weights at both 21 and 42 days with a 24.5g and 66.8g increase compared to the control, respectively. Likewise the, FCR was reduced in both the BMD and DSM 32314 treated groups at D21 and D42. This study indicates that in these conditions the DSM 32314 was able to improve FCR of broilers at least as well as the potent antibiotic growth promoter BMD compared to the control, and was even responsible for achieving the highest average weights of all the groups.

### REFERENCES

M'Sadeqa, S., Wua S., Swicka. R. A. and M. Chocta (2015). Towards the control of necrotic enteritis in broiler chickens with in-feed antibiotics phasing-out worldwide. Animal Nutrition. 1:1-11.

### Example 7: Well Diffusion Antagonism Tests with Respect to Different Pathogenic Strains

A well diffusion antagonism test with 3 different pathogens, Enterococcus cecorum DSM 20683, Streptococcus gallinaceus DSM 15349 and Streptococcus suis ATCC 43765 was performed.

E. cecorum is known to cause lameness, arthritis and osteomyelitis in broilers usually caused by an inflammation of a joint and/or bone tissue. Additional E. cecorum can cause an inflammation of the pericardium [Kense et al. 2011]. DSM 20683 was isolated from caecum of a chicken.

S. gallinaceus can cause septicaemia in poultry. The gross lesions included splenomegaly, hepatomegaly, renomegaly and congestion. Multiple areas of necrosis and/or infarction in the liver and spleen associated with valvular endocarditis were also observed [Collins et al. 2002].

S. suis is an important pathogen in pigs and one of the most important causes of bacterial mortality in piglets after weaning causing septicemia, meningitis and many other infections [Goyette-Desjardins et al. 2014]. ATCC 43765 belongs to Serological group: R; serovar 2 and was isolated from pigs.

Bacillus strains were grown in 10 mL TSBYE (30 g/l TSB+6 g/l Yeast extract) or LB-Kelly (LB-Media supplemented with trace elements solution of DSMZ media 1032) for 16 hat 37° C. and 200 rpm in 100 mL shaking flask. The pathogenic strains were grown under suitable conditions as liquid culture to an optical density of 595 nm of at least 1, then 100 μl were spread with sterile spatula on the surface of agar plates. For S. gallinaceus BHI agar plates, for E. cecorum and S. suis TSBYE agar plates are used. Three 9 mm diameter wells were cut into the dried plates. 1^{st} well was used as non-inoculated media control without culture, 2^{nd} well was inoculated with 100 uL not-inhibiting Bacillus strain (B. cereus var. toyoi, NCIMB 40112), the 3^{rd} well was inoculated with 100 uL of Bacillus subtilis DSM 32315 or DSM 17299 culture. After 24 h incubation under suitable conditions at 37° C., the zone of clearance in mm was determined measuring from the edge of the cut well to the border of the cleared lawn. Each colony was measured twice (horizontally, vertically), then averaged. The results can be found in table 7.1.

TABLE 7.1
Comparison of Bacillus licheniformis DSM 32314 and Bacillus subtilis
DSM 17299 inhibitory capacity on pathogenic strains in well diffusion
antagonism assays, values in mm clearance of pathogen.

Pathogen

| | E. cecorum | S. gallinaceus | S. suis |
|---|---|---|---|
| Probiotic | DSM 20683 | DSM 15349 | ATCC 43765 |
| DSM 32314 | 5.7 | 16.1 | 18.0 |
| DSM 17299 | 0.0 | 0.0 | 5.5 |

The data show that DSM 32314 is able to inhibit E. cecorum, S. gallinaceus and S. suis very effectively, in particular in comparison to DSM 17299.

### REFERENCES

M J Kense, W J M Landman (2011). Enterococcus cecorum infections in broiler breeders and their offspring: molecular epidemiology. Avian Pathology Vol. 40 , Iss. 6.

M D Collins, R A Hutson, E Falsen, E Ingana, M Bisgaard (2002). Streptococcus gallinaceus sp. nov., from chickens. International Journal of Systematic and Evolutionary Microbiology. 52: 1161-1164.

G Goyette-Desjardins, J-P Auger, J Xu, M Segura, M Gottschalk (2014). Streptococcus suis, an important pig pathogen and emerging zoonotic agent—an update on the worldwide distribution based on serotyping and sequence typing. Emerg Microbes Infect. 3(6):e45.

1-19. (canceled)

20. A Bacillus licheniformis strain or preparation thereof selected from the group consisting of:
a) a B. licheniformis strain as deposited under DSM 32314 at the DSMZ;
b) a mutant of the B. licheniformis strain as deposited under DSM 32314 having all identifying characteristics of the strain DSM 32314;
c) a preparation of (a) or (b); and
d) a preparation comprising an effective mixture of the strains or preparations of paragraphs (a), (b) or (c).

21. The Bacillus licheniformis strain or preparation of claim 20, wherein said strain is a mutant having all identifying characteristics of DSM 32314 and a DNA sequence identity to DSM 32314 of at least 95%.

22. The Bacillus licheniformis strain or preparation of claim 20, wherein the B. licheniformis strain further comprises:
a) a 16S rDNA sequence with a sequence identity of at least 99% to the polynucleotide sequence of SEQ ID NO: 1;
b) a yqfD sequence with a sequence identity of at least 99% to the polynucleotide sequence of SEQ ID NO: 2; and/or
c) a gyrB sequence with a sequence identity of at least 99% to the polynucleotide sequence of SEQ ID NO: 3.

23. The Bacillus licheniformis strain or preparation of claim 20, wherein the B. licheniformis strain further comprises:
a) an rpoB sequence with a sequence identity of at least 99% to the polynucleotide sequence of SEQ ID NO: 4; and/or
b) a groEL sequence with a sequence identity of at least 99% to the polynucleotide sequence of SEQ ID NO: 5.

24. The B. licheniformis strain or preparation of claim 20, wherein said strain is able to grow anaerobically.

25. The B. licheniformis strain or preparation of claim 20, wherein said strain inhibits the growth of C. perfringens.

26. The B. licheniformis strain or preparation of claim 20, wherein said strain is able to grow in the presence of 0.05 wt. % acetic acid, 0.05 wt. % propionic acid and/or 0.2 wt. % lactic acid.

27. The B. licheniformis strain or preparation of claim 20, wherein said strain comprises a cellulase activity of at least 200 mU/mL and/or a xylanase activity of at least 10 mU/mL.

28. The B. licheniformis strain or preparation of claim 20, wherein said strain is able to grow in presence of 2 mM bile.

29. A composition comprising the B. licheniformis strain or preparation of claim 20, and further comprising at least one other ingredient or compound.

30. The composition of claim 29, wherein said composition is a feed or foodstuff and said at least one other ingredient or compound is selected from the group consisting of: proteins; carbohydrates; fats; further probiotics; prebiotics; enzymes; vitamins; immune modulators; milk replacers; minerals; amino acids; coccidiostats; acid-based products;
medicines; and combinations thereof.

31. The composition of claim 29, wherein said composition is a pharmaceutical composition and said at least one other ingredient or compound is a pharmaceutically acceptable carrier.

32. A method comprising administering the Bacillus licheniformis strain or preparation of claim 20 to animals or humans or applying the strain or preparation to plants, liquids or solids in the environment.

33. The method of claim 32, wherein the Bacillus licheniformis strain or preparation is administered to an animal or human either alone, or as part of a feed or pharmaceutical composition, to improve gut health status.

34. The method of claim 32, wherein the Bacillus licheniformis strain or preparation is administered to animals either alone, or as part of a feed, to improve the health of the animals; improve the feed conversion rate of the animals; decrease the mortality rate of the animals; increase the survival rate of the animals; improve the weight gain of the animals; increase the disease resistance of the animals; increase the immune response of the animals; establish or maintain a healthy gut microflora in the animals; and/or reduce pathogen shedding through the feces of the animals.

35. The method of claim 32, wherein the Bacillus licheniformis strain or preparation is applied to manure, contaminated liquids, litter, a pit, or a manure pond to control or avoid detrimental environmental effects.

36. The method of claim 32, wherein the Bacillus licheniformis strain or preparation is applied to drinking, rearing water or to aqueous solutions to control or improve the quality of these fluids.

37. The method of claim 32, wherein the Bacillus licheniformis strain or preparation is applied to cultivated plants to treat or prevent microbial diseases.
